(19) United States
(12) Patent Application Publication
(10) Pub. No.: US 2005/0025805 A1
(43) Pub. Date: Feb. 3, 2005

(54) Osmium compounds for reduction of adverse inflammation

(76) Inventors: Adam Heller, Austin, TX (US); Sara Goldstein, Jerusalem (IL); Gideon Czapski, Jerusalem (IL)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/894,691

(22) Filed: Jul. 19, 2004

Related U.S. Application Data
(60) Provisional application No. 60/539,695, filed on Jan. 27, 2004.
(60) Provisional application No. 60/503,200, filed on Sep. 15, 2003.
(60) Provisional application No. 60/490,767, filed on Jul. 28, 2003.

(51) Int. Cl. A61K 31/41; A61F 2/00; A61L 15/00; A61K 33/24

(52) U.S. Cl. 424/423; 424/445; 424/617

(57) ABSTRACT

Reduction of adverse inflammatory reaction to an implant or a transplant, or following trauma or infection, is achieved through catalysis of dismutation of the superoxide radical anion by an osmium containing compound. Treatment diseases caused by superoxide dismutase deficiency or mutation with superoxide radical anion dismutating osmium compounds or a carbonate radical anion decay catalyzing polymeric N-oxide is also disclosed.

### CROSS-REFERENCES TO RELATED APPLICATIONS

This application claims the benefit of the following three U.S. Provisional Application Nos.: 60/490,767 (Attorney Docket No. 021821-000200US), filed on Jul. 28, 2003; 60/503,200 (Attorney Docket No. 021821-000210US), filed on Sep. 15, 2003; and 60/539,695 (Attorney Docket No. 021821-000300US), filed on Jan. 27, 2004, the full disclosures of which are incorporated herein by reference. The disclosure of this application is also related to U.S. patent application Ser. No. 10/______ (Attorney Docket No. 021821-000220US), filed on the same day as the present application, the full disclosure of which is incorporated herein by reference.

### BACKGROUND OF THE INVENTION

### FIELD OF THE INVENTION

The present invention relates generally to medical apparatus and methods for fabricating and using such apparatus. In particular, the present invention relates to the treatment, coating, or fabrication of implants, transplants, and dressings from a pharmaceutically acceptable osmium compound. The present invention also relates to the treatment, coating, or fabrication of implants, transplants, and dressings from a pharmaceutically acceptable polymeric N-oxide.

Adverse inflammatory reaction to implants and transplants. Recognition of implants or transplants as foreign bodies by the immune system triggers the recruitment of killer cells to their host tissue interface. These cells release an arsenal of chemical weapons, killing cells of the host tissue and/or of the transplant. The killing is an amplified feedback loop involving process, as the killed cells release chemotactic molecules and debris, their release further increasing the number of the recruited cells.

Adverse inflammation following trauma or infection. Inflammation, in which healthy cells of the tissue are killed, may persist after infection by a pathogen, for example of the skin, mouth, throat, rectum, a reproductive organ, ear, nose, or eye. It is desirable to terminate such inflammation as early as possible and to avoid thereby the formation of fibrotic or scar tissue. Chemotactic molecules and debris are released by cells killed by trauma, or killed by the chemical arsenal of inflammatory cells, which may persist at a site that was infected by a pathogen. Their release can lead to an amplified feedback loop, where more inflammatory killer cells are recruited. These release more of their cell killing chemicals, and more cells, releasing even more chemotactic molecules and debris, which attract even more inflammatory killer cells. The result can be the formation of physiologically non-functional fibrotic or scar tissue, and in severe cases even death. The trauma can be any event in which large numbers of cells are killed, such as exposure to excessive heat, a chemical, or sunlight.

Diseases associated with superoxide dismutase deficiency or mutation. Beyond the inflammatory diseases resulting of accumulation of killer cells and the resulting increase in the production of O₂.⁻, the published medical literature also reports evidence of diseases associated with, or resulting of, superoxide dismutase deficiency of, or defects in, often resulting of mutations, the expressed superoxide dismutase. These diseases include neurodegenerative disorders, amyotrophic lateral sclerosis, known as Lou Gehrig's disease, alcoholic liver disease, cardiovascular disease, inflammatory bowel disease, including Crohn's disease, Peyronie's disease, scleroderma and contact dermatitis. Deficiency or less than normal activity of a superoxide dismutase would also lead to an increase in the O₂.⁻ concentration and can initiate the amplified cell killing inflammatory cycle of the adverse inflammation. Thus, they could also be treated by the osmium compounds of this invention.

Coronary stents, adverse inflammation and restenosis. Vascular stents are exemplary implants. Of these, coronary stents are implanted to alleviate insufficient blood supply to the heart. Some of the recipients of coronary stents develop in-stent restenosis, the narrowing of the lumen of the coronary artery at the site of the stent, typically through neointimal hyperplasia, a result of the proliferation of fibroblasts and smooth muscle cells. (See for example, V. Rajagopal and S. G. Rockson, “Coronary restenosis: a review of mechanism and management” The American Journal of Medicine, 2003, 115(7), 547-553). The presence of macrophages and neutrophils at implants, including coronary stents, has been documented. (See, for example, Welt et al., “Leukocyte recruitment and expression of chemokines following different forms of vascular injury” Vasc. Med. 2003, 8(1), 1-7). It has also been reported that hematopoietic cells of monocyte/macrophage lineage populate the neointima in the process of lesion formation. Furthermore, macrophages have been proposed to be precursors of neointimal myofibroblasts after thermal vascular injury (Bayes-Genis et al., “Macrophages, myofibroblasts and neointimal hyperplasia after coronary artery injury and repair” Atherosclerosis, 2002, 163(1), 89-98)). According to reported theories and models (see, for example, Jeremy et al., “Oxidative stress, nitric oxide, and vascular disease” J. Card. Surg. 2002, 17(4) 324-7; Jacobson et al., “Novel NAD(P)H oxidase inhibitor suppresses angioplasty-induced superoxide and neointimal hyperplasia of rat carotid artery” Circ. Res. 2003, 92(6), 637-43; Bleeke et al., “Catecholamine-induced vascular wall growth is dependent on generation of reactive oxygen species” Circ. Res. 2004, 94(1), 37-45) by which this invention is not to be limited, O₂.⁻ is among the key risk factors for cardiovascular disease. Cardiovascular diseases, where O₂.⁻ is a risk factor, include restenosis following balloon angioplasty, atherogenesis, reperfusion injury, angina and vein graft failure.

Applications of osmium tetroxide, OsO₄. Its solution is also widely used as a biological stain, particularly in the preparation of preparation of samples for microscopy. In medicine, its solutions were injected in arthritic joints for synovectomy, the chemical removal of diseased tissue of the joint.

Chemical, non-surgical synovectomy, the chemical removal of diseased tissue of arthritic joints by injection of OsO₄. Chemical synovectomy, the chemical removal of diseased tissue of arthritic joints, has been clinically practiced since 1953. The procedure is described in the medical literature in more than 70 articles and reviews. In the procedure, OsO₄, also known as osmic acid, is injected into the diseased joint. See, for example (a) C. J. Menkes, “Is there a place for chemical and radiation synovectomy in rheumatic diseases?” Rheumatol. Rehabil. 1979,18(2), 65-77; (b) Combe et al., “Treatment of chronic knee synovitis with arthroscopic synovectomy after failure of intraarticular injection of radionuclide.” Arthritis Rheum. 1989, 32(1), 10-14; (c) Wilke and Cruz-Esteban, “Innovative treatment approaches for rheumatoid arthritis. Non-surgical synovectomy” Baillieres Clin. Rheumatol. 1995, 9, 787-801; (d) Hilliquin et al. “Comparison of the efficacy of non-surgical synovectomy (synoviorthesis) and joint lavage in knee osteoarthritis with effusions” Rev. Rhum. Engl. Ed. 1996, 63(2), 93-102; (e) Bessant et al. “Osmic acid revisited: factors that predict a favorable response” Rheumatology (Oxford). 2003, 42(9), 1036-43.

Pharmaceutical applications of osmium compounds. Hinckley, U.S. Pat. No. 4,346,216 reacted OsO₄ and osmium (VI) compounds with carbohydrates and used the resulting osmium carbohydrate complexes in pharmaceutical compositions for the treatment of heavy metal poisoning, in the treatment, by staining, of arthritic joints in mammals, and as contrast enhancing agents in X-ray diagnostic procedures. Bar-Shalom and Bukh U.S. Pat. No. 5,908,836 and U.S. Pat. No. 5,916,880 proposed the use of sulfated saccharide salts of osmium for topical treatment of the skin, for treatment or prevention of wrinkles and as X-ray contrast agents.

Relative inactivity of osmium complexes in inhibition of O₂.⁻ release from stimulated macrophages. Mirabelli et al., “Effect of Metal Containing Compounds on Superoxide Release from Phorbol Myristate Stimulated Murine Peritoneal Macrophages: Inhibition by Auranofin and Spirogermanium” The Journal of Rheumatology, 1988, 15(7), 1064-1069, investigated a series of metal complexes for their ability to inhibit the release of O₂l⁻ in the respiratory burst of macrophages. Unlike the gold complex of 2,3,4,6-tetra-O-acetyl-1-thio-β-D-glucopyranosato-S (triethylphosphine) and the germanium complex (N,N-dimethylaminopropyl)-2-aza-8,8-dimethyl-8-germanospiro-(4,5)-decane, which completely inhibited the release of O₂.⁻ at 10 μM concentration, the three osmium complexes investigated were, according to the authors, ineffective. At 10 μM concentration the % inhibition by bis(bipyridyl) dichloroosmium(II) was 2±2%; by dichlorobis(phenathroline) osmium(II) it was 24±4%; and by octamminodinitrato-(μ-nitrido)-diosmium trinitrate, at a tenfold higher, 100 μM concentration, it was 29±6 %.

Polymeric N-oxides. N-oxides are organic compounds having an oxygen covalently bound to a nitrogen, the oxygen being covalently bound to no atom other than the nitrogen. The nitrogens in N-oxides have a fractional or whole positively charge, and their oxygens, a fractional or whole negative charge. The nitrogen of an N-oxide is linked to its neighbor by four bonds, a double bond counting as two bonds. Functions I and II are N-oxide functions. Pyridine-N-oxide, III, is an example of an aromatic N-oxide. N-oxides differ from nitroxides, which are free radicals having one unpaired electron. For example, TEMPOL, IV, is a nitroxide. The nitrogen in a nitroxide is linked to its neighbors by only three bonds.

Coating of the harmful quartz particles with poly-2-vinylpyridine-N-oxide inhibits their toxicity in causing silicosis and also in oxidative DNA damage in lung epithelial cells. (R. P. F. Schins et al., “Surface modification of quartz inhibits toxicity, particle uptake, and oxidative damage in human lung epithelial cells” Chem. Res. Toxicol. 15, 1166-1173 (2002); A. M. Knaapen et al., “DNA damage in lung epithelial cells isolated from rats exposed to quartz: Role of surface reactivity and neutrophilic inflammation” Carcinogenesis (Oxford) 23(7), 1111-1120 (2002); S. Gabor, Z. Anca and E. Zugravu, “In vitro action of quartz on alveolar macrophage lipid peroxides” Archives of Environmental Health 30 (10), 499-501 (1975)).

Poly-2-vinylpyridine-N-oxide has also been used in humans as an administered drug to treat silicosis. In the treatment, doses of the polymer were administered, for example by inhalation, intravenously or by injection into muscle. (see for example, the Medline abstracts of K. V. Glotova et al., “Results of a clinical trial of polyvinoxide in silicosis” Gig. Tr. Prof. Zabol. 1981 (8), 14-7 (PMID: 7026373); J. D. Zhao, J. D. Liu and G. Z. Li “Long-term follow-up observations of the therapeutic effect of PVNO on human silicosis” Zentralbl. Bakteriol. Mikrobiol. Hyg. [B]. 1983 178(3), 259-62. (PMID: 6659745); F. Prugger, B. Mallner and H. W. Schlipkoter “Polyvinylpyridine N-oxide (Bayer 3504, P-204, PVNO) in the treatment of human silicosis” Wien. Klin. Wochenschr. 1984. 7, 96(23), 848-53 (PMID: 6396971); D. M. Zislin et al., “Therapeutic effectiveness of polyvinoxide in silicosis and silicotuberculosis” Gig. Tr. Prof. Zabol. 1985, (11), 21-5, (PMID: 4085887); T. Gurilkov and M. Stoevska “Inhalation treatment of silicosis with Kexiping” Probl. Khig. 1989, 14, 161-6 (PMID: 2635309)

### BRIEF SUMMARY OF THE INVENTION

As will be disclosed in this invention, the inventors have discovered that certain osmium compounds, such as OsO₄ and an exceptionally effective catalyst for the dismutation of the superoxide radical anion. OsO₄ is a reagent that was used used in synthetic organic chemistry, particularly in the di-hydroxylation of alkenes. Osmium containing catalysts according to the present invention reduce the likelihood of adverse inflammation. Adverse inflammation can result, for example, in the killing of cells of healthy tissue of a transplant, of host tissue near a transplant, or of host tissue near an implant. Such inflammation can also result in an unwanted change of the concentration of an analyte measured by an implanted sensor or monitor, through the consumption or generation of chemicals by inflammatory cells. Furthermore, adverse inflammation can result in reduction of the flux of nutrients and/or O₂ to cells or tissue or organ in implanted sacks, protecting the cells in the sack from the chemical arsenal of killer cells of the immune system. The cells, or tissue or organ in the sack, can replace a lost or damaged function of the human body. Adherent inflammatory cells, or fibrotic or scar cells, growing on the sack after adverse inflammatory reaction, can starve the cells in the sack.

Adverse inflammation, often associated with an inflammatory flare-up in which a large number of healthy cells of normal tissue are killed, is avoided or reduced by disruption of the feedback loop, elements of which include the release of pre-precursors of cell killing radicals by inflammatory killer cells, such as macrophages or neutrophils; release of chemotactic molecules and/or debris by the killed cells; and the recruitment of more killer cells, releasing more of the pre-precursors of the cell killing radicals.

Medical and cosmetic implants, termed here “implants”, are widely used, and novel implants are being introduced each year. Examples of the implants include vascular implants; auditory and cochlear implants; orthopedic implants; bone plates and screws; joint prostheses; breast implants; artificial larynx implants; maxillofacial prostheses; dental implants; pacemakers; cardiac defibrillators; penile implants; drug pumps; drug delivery devices; sensors and monitors; neurostimulators; incontinence alleviating devices, such as artificial urinary sphincters; intraocular lenses; and water, electrolyte, glucose and oxygen transporting sacks in which cells or tissues grow, the cells or tissues replacing a lost or damaged function of the human body. In the first of its several aspects, this invention provides materials and methods for avoidance or reduction of adverse inflammatory response in which healthy cells near the implant are killed. In its second aspect, it provides materials and methods for avoidance or reduction of the inaccuracy the measurement of the concentration of a chemical or biochemical, or a physiological parameter such as temperature, flow or pressure, by an implanted sensor or monitor, associated with an inflammatory response, where the local consumption or the local generation of a chemical or biochemical is changed by recruited inflammatory cells, or where these cells locally change a physiological parameter. In its third aspect, this invention provides materials and methods for the maintenance of a flux of nutrient chemicals, oxygen and other essential chemicals and biochemicals into implanted sacks, containing living cells or tissue, the function of which is to substitute for lost or damaged tissue, organs or cells of an animal's body, particularly the human body. If the implanted sack would cause an inflammatory response, in which normal neighboring cells would be killed, then the proliferation cells produced in the repair of the lesion would consume chemicals and reduce the influx of chemicals, such as nutrients or oxygen.

Examples of organs that are transplanted include the kidney, the pancreas, the liver, the lung, the heart, arteries and veins, heart valves, the skin, the cornea, various bones, and the bone marrow. Adverse inflammatory reaction to a transplant can cause not only the failure of the transplanted organ, but can endanger the life of the recipient.

The carbonate radical anion, CO₃.⁻, is the most potent cell killing species generated of the intermediates released by the killer cells. The hydroxyl radical, .OH, is another potent cell killer. CO₃.⁻ and .OH are generated by reactions of a common precursor, the peroxynitrite anion, ONOO⁻. The main biological source of peroxynitrite is the diffusion-limited reaction between superoxide radical anion, O₂.⁻, and nitric oxide, .NO.

The present invention provides the prevention or treatment of adverse inflammation with an osmium containing catalyst. Osmium containing catalysts, which can be locally released or can be immobilized, accelerate the decay of O₂.⁻, particularly through its dismutation to O₂ and H₂O₂. Unlike the OsO₄ used in the injected doses for chemical synovectomy, a procedure intended to remove diseased tissue by the killing of cells, the osmium containing catalysts of this invention prevent or reduce the killing of cells, and/or the associated necrosis of tissue, whether the cell or the tissue is healthy or diseased. The osmium containing catalysts can be immobilized on or near, or slowly released to, the zone to be protected against adverse inflammation. Though in many of their applications they are not systemically administered because systemic administration weakens the entire body's ability to fight pathogens, they can be systemically administered when they selectively accumulate in the zone to be treated or protected.

Examples of adverse inflammation treated or avoided through use or application of the materials and methods disclosed are inflammatory reaction to an implant, exemplified by restenosis near a cardiovascular stent; inflammatory rejection of transplanted tissue, organ, or cell; inflammation of a tissue or organ not infected by a pathogen, for example in immune, autoimmune or arthritic disease; inflammation following trauma, such as mechanical trauma, burn caused by a chemical, or by excessive heat, or by UV light, or by ionizing radiation; or persisting inflammation of the skin, mouth, throat, rectum, a reproductive organ, ear, nose, or eye following infection by a pathogen, after the population of the pathogen has declined to or below its level in healthy tissue.

According to another aspect of the present invention, poly-2-vinylpyridine-N-oxide as well as other N-oxides in polymeric coatings of implants, such as stents, or a polymeric N-oxide on, near or in a transplant, or N-oxide comprising films adsorbed on an implant, could catalyze the decay of the cell killing carbonate radical anion CO₃.⁻. Also according to this invention, when an implant or transplant is coated with, or contains, poly-2-vinyl-pyridine-N-oxide, the amplified cell killing process would be slowed or avoided.

### BRIEF DESCRIPTION OF THE DRAWINGS

FIG. 1. is a catalysis of the dismutation of O₂.⁻ by OsO₄ or its product(s). Dependence of the decay of 12 μM O₂.⁻ on the initial OsO₄ concentration, monitored by the absorbance of O₂.⁻ at 260 nm, in oxygenated pH 7.25 and 2.4 mM phosphate buffer, containing 0.02 M formate only(▪), containing 0.02 M formate with 10 μM DTPA (●), and containing instead of formate 0.2 M 2-PrOH, also with 10 μM DTPA (Δ).

FIG. 2. is a catalysis of the dismutation of O₂.⁻ by OsO₄²⁻. Dependence of the decay of 14 μM O₂.⁻ on the initial OsO₄²⁻ concentration, monitored by the absorbance of O₂.⁻ at 260 nm, in oxygenated pH 7.25 and 2.4 mM phosphate buffer, containing 0.01 M formate after the 1^{st} pulse (▪) and the 10^{th} pulse (●).

FIG. 3 is scavenging of O₂.^{-by OsO}₄²⁻. Dependence of the decay of 4 μM O₂.⁻ on the initial OsO₄²⁻ concentration, monitored by the absorbance of O₂.⁻ at 260 nm, in oxygenated pH 7.25 and 2.4 mM phosphate buffer, containing 0.01 M formate.

FIG. 4 is measured first-order rate constants for the decay of 4 μM CO₃.⁻ as a function of [PVPNO] at pH 10.0, 0.1 M carbonate, 25° C.

### DETAILED DESCRIPTION OF THE INVENTION

Terms and Definitions. Adverse inflammation or adverse inflammatory reaction is an inflammation other than inflammation to fight pathogens or mutated cells. Often large numbers of normal cells die in adverse inflammation.

Implant means a component, comprising man-made material, implanted in the body. The man made material can be a thermoplastic, a thermosetting or an elastomeric polymer; a ceramic; a metal; or a composite containing two or more of these.

Transplant means a transplanted tissue, a transplanted organ or a transplanted cell. The transplant can be an allograft or a xenograft. An allograft is a tissue or an organ transplanted from one animal into another, where the donor and the recipient are members of the same species. A xenograft is a tissue or an organ transplanted from one animal into another, where the donor and the recipient are members of different species. The animals are usually mammals, most importantly humans.

Chemotaxis is the migration of killer cells to the source of chemicals and/or debris from damaged or dead cells usually damaged or killed by killer cells.

Killer cells are either cells generating chemicals or biochemicals that kill cells, or progenitors of the actual killer cells. The killer cells are usually white blood cells or cells formed of white blood cells. Macrophages, giant cells and cells formed of macrophages, as well as neutrophils, are examples of killer cells. The macrophages are said to be formed of monocytes in the blood.

Chemotactic recruitment means causing the preferred migration of killer cells, or progenitors of killer cells, to the implant or to the transplant and their localization in or near it. Chemicals and/or debris from killed cells of the tissue hosting the implant or the transplant, or from killed cells of the transplanted tissue or organ is chemotactic, meaning that the released molecules and/or debris recruits more killer cells or progenitors of killer cells.

Programmed cell death is normal orchestrated cell death in which the dead cell's components are so lysed or otherwise decomposed that few or no chemotactic molecules and/or debris are released.

Immobilized catalyst and insoluble catalyst mean a catalyst that is insoluble, or that dissolves, or that is leached, very slowly. A very slowly dissolving or leached catalyst is a catalyst less than half of which dissolves in one day, or is otherwise leached in one day, by a pH 7.2, 0.14 M NaCl, 20 mM phosphate buffer solution at 37° C. in equilibrium with air.

Plasma means the fluid bathing the implant or the transplanted tissue, organ or cell, and/or the intercellular fluid bathing the cells of the transplanted tissue, organ or cells.

Near the implant or near the transplant means the part of the tissue or organ hosting the implant or the transplant, located within less than 5 cm from the implant or the transplant, preferably within less than 2 cm from the implant or the transplant and most preferably within less than 1 cm from the implant or the transplant.

Permeable means a film or membrane in which the product of the solubility and the diffusion coefficient of the permeating species is greater than 10⁻¹¹ mol cm⁻¹ s⁻¹ and is preferably greater than 10⁻¹⁰ mol cm⁻¹ s⁻¹ and is most preferably greater than 10⁻⁹ mol cm⁻¹ s⁻¹.

Hydrogel means a water swollen matrix of a polymer, which does not dissolve in an about pH 7.2-7.4 aqueous solution of about 0.14 M NaCl at about 37° C. in about 3 days. It contains at least 20 weight % water, preferably contains at least 40 weight % water and most preferably contains at least 60 weight % water. The polymer is usually crosslinked.

Dressing means a covering for a wound or surgical site, typically composed of a cloth, fabric, synthetic membrane, gauze, or the like. Dressings will also include gels, typically cross-linked hydrogels, which are intended principally to cover and protect such wounds, surgical sites, and the like.

Pharmaceutically acceptable means that the implant, dressing, and/or osmium compound of the present invention is non-toxic and suitable for use for the treatment of humans and animals. Such pharmaceutically acceptable structures and compositions will be free from materials which are incompatible with such uses.

Topical composition means an ointment, cream, emollient, balm, salve, unguent, or any other pharmaceutical form intended for topical application to a patient's skin, organs, internal tissue sites, or the like.

The present invention provides treatment and structure to avoid or reduce adverse inflammation in which healthy cells of normal tissue are killed. Its specific purpose includes avoidance, reduction, or alleviation of (a) adverse inflammatory reaction to implants, exemplified by restenosis near cardiovascular stents; (b) inflammatory rejection of transplanted tissues or organs or cells; (c) inflammation of a tissue or organ when not infected, for example in immune disease, autoimmune disease, arthritic disease, neurodegenerative disorders, amyotrophic lateral sclerosis known as Lou Gehrig's disease, alcoholic liver disease, cardiovascular disease, inflammatory bowel disease including Crohn's disease, Peyronie's disease, scleroderma and contact dermatitis; (d) inflammation following trauma and/or burn such as burn caused by excessive heat and/or UV and (e) inflammation of the skin, mouth, throat, rectum, a reproductive organ, ear, nose, or eye following infection.

Recognition and the recruitment of inflammatory killer cells. Inflammation is generally associated with the recruitment of white blood cells, exemplified by leucocytes, such as neutrophils and/or monocytes and/or macrophages. The white blood cells secrete pre-precursors of potently cell killing oxidants. According to theoretical models, by which this invention is not to be limited, the rejection of transplants involves recognition, usually by lymphocytes, resulting, after multiple steps, in the killing of some cells of the transplant, then in the eventual chemotactic recruitment of killer cells by debris of the killed cells, and the killing of more cells by oxidants generated by the killer cells. The sequence of recruitment of killer cells, the killing of cells by the oxidants they secrete, the killing of more cells, the release of chemotactic chemicals and/or debris and the recruitment of an even greater number of killer cells constitute an amplified feedback loop.

The arsenal of killer cells. The cell-killing arsenal of the inflammatory cells, such as macrophages and neutrophils, includes two radicals, the superoxide radical anion, O₂.⁻ and nitric oxide, .NO. Superoxide radical anion is produced in the NADPH-oxidase catalyzed reaction of O₂ with NADPH. Nitric oxide is produced by the nitric oxide synthase (NOS) catalyzed reaction of arginine. The NOS of inflammatory cells is iNOS, inducible nitric oxide synthase. These radicals are relatively long-lived in the absence of scavenging reactants or enzymes accelerating their reactions, their half live equaling or exceeding a second. For this reason, their diffusion length, L, which is the square root of the product of their half life, τ_{1/2}, and their diffusion coefficient, D, which is about 10⁻⁵ cm² sec⁻¹, can also be long, equaling or exceeding 30 μm, a distance greater than the distance between the centers of large cells. Thus, the pre-precursors secreted by nearby killer cells can reach and enter nearby tissue cells. The oxidant precursors, formed of the pre-precursors O₂.⁻ and NO, include the also long lived ONOO⁻ and H₂O₂. At the physiological pH of 7.2-7.4, and in absence of enzymes accelerating their reaction, such as catalase or peroxidase in the case of H₂O₂, their τ_{1/2}≧1 second, and their L≧30 μm. The ONOO⁻ precursor reacts with CO₂, which abounds in tissues and cells, to form the potently oxidizing CO₃.⁻ and nitrogen dioxide radical, .NO₂ H₂O₂ may react with transition metal complexes to form the hydroxyl radical, .OH, which reacts rapidly with any oxidizable matter, including glucose, and or proteins, at the site of its formation, and can even react with HCO₃⁻, to form CO₃l⁻. The τ_{1/2} of CO₃.⁻ is about 1 millisecond, and its L is about 1 μm. Thus, after a precursor enters a cell and reacts to form CO₃.⁻, the CO₃.⁻ lives long enough to diffuse across distances approaching or equaling the dimension of the cell, allowing it to oxidize any of its oxidizable components. This makes it the premier killer of cells.

Potently cell killing CO₃.⁻ generated from its ONOO⁻ precursor and the importance of superoxide dismutase and/or superoxide dismutase mimics in reducing the killing of cells by CO₃.⁻. The nature of the chemicals secreted by white blood cells, termed here pre-precursors, and the chemicals formed of these pre-precursors, termed here precursors, as well as the potently cell killing chemicals formed of the precursors, is known. The white blood cells generate two important pre-precursors, O₂.⁻ and .NO. O₂.⁻ is believed to be generated by NADPH oxidase-catalyzed reduction of O₂. .NO is believed to be generated through nitric oxide synthase, NOS, catalyzed oxidation of arginine. The NOS of white blood cells is believed to be inducible nitric oxide synthase, iNOS.

The peroxynitrite anion, ONOO⁻, which is formed through Reaction 1, is a precursor of highly toxic entities.
O₂.⁻+.NO→ONOO⁻k₁=5×10⁹M⁻¹s⁻¹ (1)

Peroxynitrite ion is fairly stable but its conjugate peroxynitrous acid (ONOOH, pKₐ=6.6) decomposes rapidly; isomerization to nitrate is the major decay route in acidic media. On its way to NO₃⁻, a significant portion (˜28%) of ONOOH produces the hydroxyl and nitrogen dioxide radicals (Scheme 1).

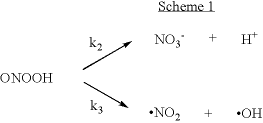

According to accepted models, cell killing CO₃.— is generated from ONOO⁻ through its rapid reaction with CO₂ (Scheme 2). The half live (τ_{1/2}) of their product

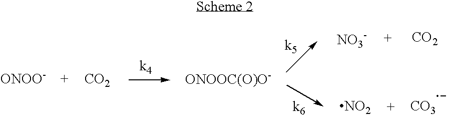

ONOOC(O)O⁻, is estimated to be shorter than 100 ns. Consequently, this adduct decomposes to non-reactive NO₃⁻ and CO₂, or to highly reactive and toxic CO₃.⁻ and .NO₂ before it can react with components of biological systems.

Application of the reported values at 38° C. for (k₂+k₃)=5.3 s⁻¹, k₄=5.3×10⁴ M⁻¹s⁻¹ and the concentrations of CO₂ in intracellular ([HCO₃⁻]=12 mM) and in interstitial fluids ([HCO₃⁻]=30 mM), leads to the conclusion that the reaction of peroxynitrite with CO₂ is the dominant pathway of peroxynitrite consumption in biological systems.

The hydroxyl radical is so reactive that it reacts nearly non-selectively with any molecules at the site of its formation. On the other hand, CO₃.⁻ is less reactive and is, therefore, more selective. Thus, according to the best available models, by which this invention is not to be limited, the most important cell killing species formed is probably CO₃.⁻ with .NO₂ as an also cell killing, but less potent species.

The amount of O₂.⁻ available for generating ONOO⁻ is reduced in the presence of superoxide dismutase, SOD, which catalyzes the dismutation of O₂.⁻ (Reaction 2) very efficiently.
2 O₂.⁻+2 H⁺→H₂O₂+O₂ (2)

Hence, efficient removal of O₂.⁻ prevents the formation of ONOO-, and thereby the killing of cells by CO₃.⁻ and/or .NO₂.

O₂.⁻ and adverse inflammation. Adverse inflammatory response to chronic implants or transplants, leading, for example, to restenosis at sites of cardiovascular stents is associated with downstream products of reactions of the superoxide radical anion. The in vivo catalytic destruction of this radical could alleviate or prevent undesired inflammation, inflammatory response to implants exemplified by restenosis, and/or acute inflammatory rejection of transplanted tissue or organs.

Adverse inflammation near implants. Inflammatory killer cells, like macrophages and neutrophils, evolved to destroy organisms recognized as foreign. They persistently try to destroy implants and can cause restenosis in stented blood vessels. They adhere to and merge even on implants said to be biocompatible, often forming large macrophage covered areas. Their presence on chronic implants usually leads to a permanent, clinically acceptable low level of inflammation, though in part of the orthopedic and other implants periodic adverse inflammatory flare-ups do occur.

The peroxynitrite anion precursor of the cell killing CO₃.⁻ and .NO₂ is produced by the combination of two macrophage-produced radicals, .NO and O₂.⁻. Nitric oxide is a short-lived, biological signal transmitter. By itself it is not a strong oxidant. O₂.⁻ is also not a potent oxidant, behaving in some reactions as a reducing electron donor. The half lives of .NO and O₂.⁻ can be long, >1 second. The product of their combination, ONOO⁻, oxidizes a large variety of biomolecules mostly indirectly through the formation of highly oxidizing radicals as intermediates, namely .OH/CO₃.⁻ and .NO₂.

When cells die naturally, by the orchestrated process of apoptosis, their decomposition products are not chemo-attractants of macrophages. In contrast, when cells are killed by the products of peroxynitrite, the chemicals and/or debris released are chemotactic for (chemically attract, or “recruit” more) macrophages. As a result a feedback loop, a flare up in which many cells are killed, can result. The killing of many cells can produce a lesion. As the killing of more cells leads to more debris and to the recruitment of even more macrophages, and as more macrophages are recruited, the damage is amplified and the size of the lesion is increased. The body's subsequent repair of the lesion can lead to the proliferation of cells and can underlie stent-caused restenosis. This self-propagating, increasingly destructive process can be avoided by using the described materials, and disrupted, slowed, alleviated, or stopped by the disclosed O₂.⁻ dismutation and/or ONOO⁻ isomerization catalysts and/or catalytic destruction of CO₃.⁻.

The catalyst can be coated on implants prior to their implantation, incorporated in the coating of the implant, or incorporated in the tissue proximal to the implant. Two groups of catalysts are particularly useful. The first, for O₂.⁻ dismutation, contains osmium. The second, for ONOO⁻ isomerization, are immobilized ONOO⁻ and/or NO₃.⁻ permeable hydrogels, containing porphyrins and phthalocyanines of transition metals, particularly of iron and manganese, known to catalyze the peroxynitrite to nitrate isomerization.

The third, for CO₃.⁻ destruction, are immobilized CO₃.⁻ and/or HCO₃⁻ permeable hydrogels, containing porphyrins of transition metals, and/or derivatives of cyclic N-oxide and/or N-oxyl and/or hydroxylamines. Examples of these, particularly of manganese porphyrins, were described, for example, by G. Ferrer-Sueta et al in J Biol. Chem. 2003, 278, 27432-27438, and examples of nitroxides, N-oxides and or N-oxyl and/or hydroxylamines were described by co-applicant S. Goldstein et al, Chem. Res. Toxicol. 2004, 17, 250-257.

Polymeric pyridine-N-oxides. According to the present invention, poly-2-vinylpyridine-N-oxide, as well as other N-oxides in polymeric coatings of implants, such as stents, or a polymeric N-oxide on, near or in a transplant, or N-oxide comprising films adsorbed on an implant, could catalyze the decay of the carbonate radical anion CO₃.⁻. According to this invention, when an implant or transplant is coated with, or contains, poly-2-vinyl-pyridine-N-oxide, the amplified cell killing process would be slowed or avoided. The thickness of the polymeric N-oxide containing film or layer on the implant or in or near the transplant would be such that it will be clinically useful. Films of one monolayer thickness could already be useful. The preferred thickness would be between about 10 nm and about 1 mm, a more preferred thickness would be between about 10 nm and about 100 μm, and the most preferred thickness would be between about 100 nm and about 20 μm.

Among the N-oxides of this invention compounds where the nitrogen is part of a ring are preferred and compounds where it is part of an aromatic ring are most preferred. Polymers having N-oxide functions in their repeating are preferred. The N-oxides are preferably immobilized in the coating of the implant, such as the stent, and in or at the surface of the transplant. In general, poly-2-vinylpyridine-N-oxide, as well as other N-oxides, as well as other coatings or compounds known to reduce the toxicity of quartz particles are expected to prevent, or reduce the frequency, of in stent-restenosis in coronary stents, as well as adverse inflammatory effects and cell damage at other implants and at transplants. When in blood or exposed to flowing blood, it is preferred that the catalyst, whether an N-oxide or other, be immobilized and not be leached, or be leached only very slowly, because the rapidly circulating blood in a blood vessel, such as the coronary artery in the case of a coronary stent, or rapidly circulating blood in some transplants, exemplified by kidney transplants, could rapidly strip the catalyst. The N-oxide in the coatings, whether poly-2-vinylpyridine-N-oxide or another polymer bound N-oxide, could be such that the N-oxide would not be leached when the leaching solution is an unstirred, approximately pH 7.2 0.02M phosphate buffered saline solution, containing about 0.14 M NaCl at about 37° C. and the test for leaching is about 2 weeks long. Alternatively, the N-oxide coatings could be such that some of the N-oxide in the coating, preferably not more than about 10 % of the N-oxide, would be leached when the unstirred leaching solution is an approximately pH 7.2 0.02M phosphate buffered saline solution, containing about 0.14 M NaCl, at about 37° C., and the test for leaching is about 2 weeks long. In general, it is preferred that the catalyst be immobilized, not be leached, and remain active for about 2 weeks or more, preferably 1 month or more, and most preferably for about 2 months or more, when in antibiotic stabilized serum at 4° C.

Poly(2-vinylpyridine-N-oxide). Four repeating units (mers) is shown below:

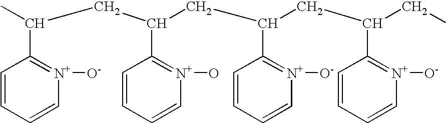

A variety of water soluble, polymeric N-oxides, wherein the nitrogen is part of an aromatic or heterocyclic ring are useful in the coating of implants or for incorporation in or at transplants. Their aromatic or heterocyclic rings can have five or six ring atoms. Six membered aromatic ring N-oxides and five or six membered heterocyclic ring N-oxides are generally preferred. The polymeric N-oxides can be water-soluble and they could be irreversibly adsorbed from an aqueous solution, or co-deposited and cured with a crosslinker to form a coating. The preferred polymeric N-oxides can have molecular weights from about 3000 to about 100,000,000; the preferred molecular weights are between 5000 and 5000000, with the range 10000 to 500000 being most preferred. In the case of stents, the thickness of the crosslinked polymer coatings would be such that when in equilibrium with plasma at 37° C. the volume occupied by the coating would be less than 10% of the internal volume of the expanded stent, preferably less than 3% of the internal volume of the expanded stent and most preferably less than 1 % of the internal volume of the expanded stent. The polymeric N-oxide could be crosslinked, for example, with di-, tri-, or poly-epoxides, such as polyethyleneglycol diglycidyl ether.

The family of polymeric N-oxides includes, for example, poly(2-vinylpyridine-N-oxide), poly(4-vinylpyridine-N-oxide), poly(3-methyl-2-vinylpyridine-oxide), and poly(ethylene 2,6-pyridinedicarboxylate-oxide). The N-oxides, whether polymeric or monomeric, could have alkylated or alcohol-functionalized, for example —CH₂OH functionalized, rings; or halide-substituted rings; or thiol or amine functionalized rings, or carboxylate functions, exemplary functions being —Cl, —CH₂Cl, —CH₂NH₂, —CH₂SH, —COOH. The —CH₂NH₂ and the —CH₂SH functions are known to add at ambient temperature and in aqueous solutions to double bonds by the Michael reactions. In these, monomers or polymers having for example, an —C(R)═C(R′)—C(═O)— function could combine with an exemplary —CH₂NH₂ or —CH₂SH functions. This would allow the crosslinking of the polymeric N-oxide molecules. The monomeric N-oxides functionalized with —CH₂SH or —CH₂NH₂ functions, also add, by Michael reaction, to acrylic or similar functions, making acrylate, methacrylate and related function carrying polymers catalytic.

Films of the polymeric N-oxide could be conveniently formed on the implant by adsorption on the surface oxide layer of its metal or ceramic. Typically, the concentration of the polymeric N-oxide in the aqueous solution from which it could be adsorbed would be about 0.1-10 weight %. The film could also be formed by co-adsorbing the polymeric N-oxide and its crosslinker from an aqueous solution, in which the two are co-dissolved. An exemplary crosslinker would be poly(ethylene oxide)diglycidyl ether of about 400 molecular weight. For this crosslinker and for poly(2-vinylpyridine-N-oxide) the preferred polymer/crosslinker weight ratio would be between about 30:1 and about 5:1, a weight ratio of about 25:1 and about 10:1 being most preferred.

The polymer coating could be applied, for example, after pre-cleaning with isopropanol the stent or other implant, rinsing with de-ionized water, drying, reactively oxidizing for 10 min in an RF(50-150 W) plasma furnace at 1-2 mm Hg oxygen pressure, to oxidize the organic surface impurities, then applying the aqueous polymer, or polymer with crosslinker solution, by a method such as dipping, spraying, or brushing, then allowing the film to dry or cure, usually at ambient temperature, for at least 24 h.

Proposed ethiology of restenosis. According to this invention, restenosis, the in-stent proliferation of fibroblast and smooth muscle cells, involves an inflammatory process, resulting in the killing of healthy cells of the coronary artery. The killing of the cells results in a lesion, which is repaired not by growth of normal endothelial cells, but by proliferating fibroblasts and smooth muscle cells, the cells causing the narrowing of the lumen of the artery in neointimal hyperplasia. The neointimal hyperplasia causing process may start, for example, with the recruitment of a few phagocytes, such as macrophages and neutrophils, by corroding microdomains, usually microanodes, of the transition metal comprising stent alloy, or by residual protruding features of the stent, particularly by features having dimensions and shapes resembling bacteria. Next, some of the chemical zones and/or protruding topographic features of the surface of the stent are covered by recruited phagosomes. In these, potent cell killing species, particularly CO₃.⁻ radicals, are generated from their macrophage and/or neutrophil generated ONOO⁻ precursor, eventually killing the phagosome. Its killing results in the release of chemotactic molecules and/or debris, which attract more macrophages and/or neutrophils. As a result, the surface of the stent becomes densely populated by these cells. For individual killer cells, the concentrations of O₂.⁻ and .NO, the secreted pre-precursors of cell killing radicals, declines with the cube of the distance from the cell. Hence, individual macrophages or neutrophils are ineffective killers of cells other than the cells they phagocytize. In contrast, when a surface is densely populated by macrophages or leucocytes, their concentration declines linearly with the distance from the macrophage or leucocyte covered surface. Hence, the radicals combine to form, with higher yield, ONOO⁻, the precursor of the highly toxic, cell killing, CO₃.⁻, to less extent .NO₂ and/or the potently oxidizing, possibly also formed, .OH. The killing of a massive number of the cells by CO₃.⁻ and/or .OH results in a lesion. The imperfect repair of the lesion by proliferating fibroblasts and smooth muscle cells results in restenosis, the narrowing of the lumen of the artery.

Adverse inflammation in the acute rejection of transplants. As discussed above, white blood cells can kill cells of transplants. Their presence on transplants can cause a permanent, low-level inflammation, which can be tolerated and is clinically acceptable. In part of the transplants, it causes, however, inflammatory flare up and necrosis. The amplified cycle underlying the flare up and/or necrosis usually involves the generation of, and the killing of cells by, strong oxidants exemplified by products of reactions of the peroxynitrite anion, particularly CO₃.⁻ and/or .OH.

Treatment of diseases resulting of superoxide dismutase deficiency, defect or mutation. Because the osmium containing compounds of this invention accelerate the decay of O₂.⁻, most probably its dismutation to O₂ and H₂O₂, and because the absence of systemic toxicity of OsO₄ has been established through more than 50 years of its use in synovectomy of arthritic joints, diseases associated with or resulting of superoxide dismutase deficiency, defect or mutation could be treated with the osmium containing compounds of this invention. Examples of diseases resulting of or associated with deficiency, defect or mutation of superoxide dismutase include neurodegenerative disorders, amyotrophic lateral sclerosis known as Lou Gehrig's disease, alcoholic liver disease, cardiovascular disease, inflammatory bowel disease including Crohn's disease, Peyronie's disease, scleroderma and contact dermatitis.

Catalysts coated on and/or slowly released from coatings on implants or transplants. Osmium containing catalysts accelerating the decay of the concentration of O₂.⁻, for example by its dismutation to O₂ and H₂O₂, and hydrogel-bound catalysts of the isomerization of OONO⁻ to NO₃.⁻, and efficient catalyst for CO₃.⁻ destruction are disclosed. The catalysts are intended to prevent, reduce or alleviate adverse inflammation near implants, or the inflammatory rejection of transplants. Preferably, the catalysts are immobilized in, on, or near the implant, or the transplanted tissue, organ, or cell.

These catalysts accelerate a reaction wherein OONO⁻ precursor or the O₂.⁻ pre-precursor of cell killing CO₃.⁻ and/or OH is consumed in, on, or near the implant or the transplanted tissue, organ, or cell is reduced, without substantially affecting the concentration of OONO⁻, or O₂.⁻, in tissues or organs remote from the implant or transplant. Preferably, the catalyst affects the concentration of OONO⁻, or O₂.⁻ locally, not systemically. The preferred catalysts do not affect the concentrations of OONO⁻ or O₂.⁻ in organs or tissues at a distance greater than about 5 cm from the implant or transplant, preferably do not affect these at a distance greater than about 2 cm from the implant or transplant, and most preferably they do not affect these at a distance greater than about 1 cm from the implant or transplant.

The model of the amplified cell killing cycle, disrupted by the immobilized catalysts of this invention, by which this invention is not being limited, is the following. The CO₃.⁻ radical, and the .OH radical, are cell-killing oxidants. When a cell dies naturally, by the orchestrated process of programmed cell death, its decomposition products are not chemo-attractants of macrophages or other killer cells. In contrast, when a cell is killed by a product of a reaction of ONOO⁻, molecules released by, or debris produced of, the dead cells is chemotactic for (chemically attracts, or “recruits” more) killer cells and/or their progenitors, such as monocytes, macrophages and/or neutrophils. The greater the number of the cells killed, the greater the number of killer cells or killer cell progenitors recruited by the chemotactic molecules released from, and/or chemotactic debris from, the dead cells. The greater the number of, or the coverage of the transplant by, debris-recruited macrophages, the greater the rate of local generation of the two precursors of which the peroxynitrite killer anions are spontaneously formed, which are nitric oxide (NO) and the superoxide radical anion (O₂.⁻). The result is a cell death-amplified, peroxynitrite anion-mediated, feedback loop, resulting in a flare up in which more of the transplanted cells are killed. This self propagating, progressively more destructive cycle can be slowed or prevented by reducing the local concentration of peroxynitrite anions through an immobilized catalyst accelerating their isomerization, or accelerating the decay of their O₂.⁻ precursor.

The catalyst can be immobilized on the implant prior to implantation. Optionally, it can be slowly released after implantation. Alternatively, it can be in a hydrogel immobilized on the surface of the implant. The preferred hydrogels are permeable to OONO⁻ and/or to NO₃⁻ and/or to O₂.⁻ and/or H₂O₂. The catalyst can be incorporated in, on, or near a transplant after transplantation, or it can be incorporated in or on the transplant after its removal from the donor but prior to transplantation in the recipient. The catalyst can be a polymer-bound molecule or ion, bound within the polymer by electrostatically, and/or coordinatively and/or covalently and/or through hydrogen bonding, and/or through hydrophobic interaction. The preferred polymers, to which the catalyst is bound, swell, when immersed in a pH 7.2 solution containing 0.14 M NaCl at 37° C. to a hydrogel.

The immobilized, or slowly leached, catalyst can lower near the implant, or near the transplant, or near an inflamed organ, such as the skin after it is burned, the local concentration of OONO⁻ through its isomerization reaction OONO⁻→NO₃⁻, or through any reaction of its precursor O₂.⁻ other than combination with .NO, whereby OONO⁻ would be formed. Preferably, the catalyst lowering the O₂.⁻ concentration contains osmium and most preferably it dismutates O₂.⁻ through Reaction 2.

Osmium containing catalysts. The osmium containing catalysts accelerate the decay of the concentration of O₂.⁻ through acceleration of any reaction in which O₂.⁻ is consumed, other than the combination of O₂.⁻ with .NO. The osmium containing catalysts accelerate preferably Reaction 2, the dismutation of O₂.⁻ to O₂ and H₂O₂.

The preferred osmium containing catalysts contain oxygen, or a function, such as a halide anion, exchanged in the body by an oxygen containing molecule, ion or radical, like water, or hydroxide anion, or O₂.⁻, so that a bond between osmium and oxygen atom is formed. At least part of the oxygen of the catalyst is directly bound to osmium. The bond between the osmium and the oxygen can be electrostatic, also termed ionic, and/or covalent, and/or coordinative. Exemplary molecules and ions that are useful catalysts are OsO₄, where the formal oxidation state of osmium is (VIII) and the bonding between the molecules is non-ionic; salts like Ba₅(OsO₆)₂, where the formal oxidation state of osmium is (VII); salts like K₂OsO₄, BaOsO₄.4H₂O, BaOsO₄.2H₂O, BaOsO₄, Ba₂OsO₅, Ba₃OsO₆, Ca₂Os₂O₇, CuOsO₄ or ZnOsO₄, where the formal oxidation state of osmium is (VI), or a polymer, such as polyvinyl pyridine reacted osmium tetroxide, where the formal oxidation state of osmium is also (VI); salts like Ca₂Os₂O₇, where the formal oxidation state of osmium is (V); Salts like (NH₄)₂OsO₃, CaOsO₃, or SrOsO₃, or metallic oxides like OsO₂, or hydrated, or non-metallic OsO₂. nH₂O with n ≧0.5, where the formal oxidation state of osmium is (IV); hydrated Os (III) salts, like OsCl₃.nH₂O where n≧3 or OsBr₃.nH₂O where n≧3; hydrated Os (II) salts, like OsCl₂.nH₂O or OsBr₂.nH₂O where n ≧4, and metallic osmium, whether elemental or alloyed, under conditions where it could corrode enough to produce an about 1 nM concentration of a dissolved osmium species in the solution it contacts. The catalytic compounds and salts can be non-stoichiometric. The osmium compounds are commercially available from Alfa Asear, Ward Hill Mass. or from Sigma Aldrich, Milwaukee, Wis., or can be prepared by reported methods. Scholder and Schatz (Angewandte Chemie 1963, 75, 417) prepared Os(VII) as Ba₅(OsO₆)₂ and Os(VI) as BaOsO₄.4H₂O, as well as Ba₂OsO₅ and as Ba₃OsO₆. Bavay (Revue de Chimie Minerale, 1975, 12(1), 24-40) showed that Ba(NO₃)₂ precipitates from osmate solution BaOsO₄.2H₂O. Chihara (Proceedings of the 5th International Conference on Thermal Analysis, V. B. Lazarev and I. S. Editors, Heyden, London, UK (Publisher), 1977, 273-5) showed that in air CaOsO₃ reacts with O₂ to give Ca₂Os₂O₇ at 775-808° C., which decomposes at 850-1000° C. to Ca₂Os₂O_{6.5}, a non-stoichiometric compound; SrOsO₃ is converted at 970-1020° C. to Sr₂Os₂O_{6.4±0.2}, also a non-stoichiometric compound; BaOsO₃ is oxidized to BaOsO₄ at 830-900° C. Gilloteaux and Naud, (Histochemistry, 1979, 63(2), 227-43) described the formation of CUOsO₄ and ZnOsO₄; and Shaplygin and Lazarev (Zhurnal Neorganicheskoi Khimii 1986, 31(12), 3181-3) described the formation of BaOsO₄ and BaOsO₃.

While catalysts in which the osmium is bound to at least three oxygens are preferred, and those where osmium is bound to at least four oxygens are most preferred, compounds of osmium, including complexes of osmium, wherein the oxygen is linked to at least two oxygen atoms, or where at least two of the ligands are exchanged under physiological conditions with a small oxygenated species like water or O₂.⁻, are also useful. The readily exchanged ligand can be, for example, a halide, ammonia, an N-oxide, a phosphine-oxide, or a sulfoxide.

The preferred solubility of the catalytic osmium compound is greater than 10⁻⁹ M, and a solubility exceeding 10⁻⁸ is most preferred. In implants or transplants, where very high solubility that could lead to rapid leaching of the surface-immobilized catalyst by fluids of the body, it is preferred that the concentration of the osmium containing molecule or ion in serum equilibrated with the source of the osmium compound at 37° C. be less than about 10⁻⁴ M, and it is most preferred that it be less than about 10⁻⁵ M.

The preferred osmium containing catalysts for O₂.⁻ dismutation are transiently or permanently be immobilized on the surface of the implant or in the plasma contacting surface zone of the transplant and/or in the host tissue near the transplant, or in a membrane surrounding the transplant. The most preferred catalytic oxides are those of osmium. These oxides include osmium tetroxide or can be formed of osmium tetroxide or the hydrolysis of osmium halides, can be formed, for example, by reduction of liquid or liquid osmium tetroxide.

The key measure of the performance of the osmium catalyst in a homogeneous solution is the rate constant k_{cat}. The rate of the O₂.⁻ elimination reaction, of importance in the suppression of adverse inflammation, which is the rate of the decay of the O₂.⁻ in the presence of the catalyst. As seen in the Examples, in a pH 7.25 buffer containing 2.4 mM phosphate at 25° C., k_{cat} is uniquely and surprisingly high for OsO₄. The k_{cat} of OsO₄ is (1.02±0.08)×10⁹ M⁻¹s⁻¹, about ⅓^{rd} of k_{cat} of the natural enzyme, copper-zinc superoxide dismutase, CuZn—SOD. Because the molecular weight of the CuZn—SOD is 32 kDa and that of OsO₄ is only 254 Da, the specific activity, which is the rate of decay of O₂.⁻ per unit weight of catalyst, is 42 times faster for OsO₄ than it is for CuZn—SOD, making it the best weight-based catalyst for the elimination of 02-, apparently by its dismutation. The specific gravimetric activity (specific activity per unit weight) of OsO₄ is about 1.02×10⁹/254=4.0×10⁶ M⁻¹ s⁻¹ g⁻¹; that of CuZn—SOD is only about 9.4×10⁴ M⁻¹ s⁻¹ g⁻¹. Because the density of OsO₄ is about 4.9 g cm⁻³, while the density of proteins is about 1.4 g cm⁻³, the specific volumetric activity (specific activity per unit volume) of OsO₄ is about 2.0×10⁷ M⁻¹ s⁻¹ cm⁻³, while that of CuZn—SOD is only about 1.4×10⁵ M⁻¹ s⁻¹ cm⁻³, a 135 fold advantage in the volume of required catalyst. Therefore, in an exemplary homogeneous catalyst eluting stent or other implant, the OsO₄ catalyst required would weigh about 42 times less, and its volume would be about 135 times smaller, greatly simplifying the structure and facilitating the manufacture of a the catalyst eluting implant, transplant or dressing, for example on the skin.

In the least active osmium containing catalysts, Os²⁺ was complexed by three 2,2′-bipyridines, or by three 2,2′-(4,4′-dimethylbipyridines), the soluble ions being Os(bpy)₃²⁺ and Os(dimebpy)²⁺, which would be oxidized in the oxygenated solution used to the Os(bpy)₃ ^{2+/3+} and Os(dimebpy)^{2+/3+} redox couples. For these complexes k_{cat} was too low to be measured.

In general, k_{cat} is higher for the higher valent osmium compounds. Therefore, catalysts in which the formal valence of osmium is greater than 4 are preferred, and catalysts in which the formal valence of osmium is greater than 6 are most preferred. Although, as seen in the Examples, k_{cat} of solutions of Os²⁺ or Os³⁺ salts is much lower than that of OsO₄, the catalytic activity of the Os²⁺ or Os³⁺ salts increases drastically when repeatedly exposed to pulses of reactive oxygenated species like O₂.⁻, establishing that the O₂.⁻ pre-precursor and the OONO⁻ precursor of the inflammatory cell-killing CO₃.⁻ or .OH can activate the catalytically less active lower-valent osmium species. Therefore, in spite of their lesser initial catalytic activity, it is expected that in the environment of the killer cells, the lower-valent osmium catalysts will be activated to become potent catalysts of acceleration of the decay of the concentration of O₂.⁻, most probably through Reaction 2, its dismutation.

Immobilized and/or slowly dissolving osmium catalysts can be used in order to maintain a high rate of catalytic O₂.⁻ conversion. The rate to be should be adequate for the half-life of O₂.⁻ to be reduced to less than about 10 seconds, and preferably less than about 1 second. For an exemplary catalyst with k_{cat}=10⁸M⁻¹ s⁻¹, the catalyst concentration should exceed in the tissue or zone to be shielded from the O₂.⁻ of killer cells, 10⁻⁹ M and should preferably exceed 10⁻⁸ M. The concentration of OsO₄, with k_{cat}≈10⁹M⁻¹ s⁻¹, should exceed about 10⁻¹⁰ M and should preferably exceed about 10⁻⁹ M. For a less effective catalyst, with k_{cat}=10⁸ M⁻¹ s⁻¹, the concentration should be greater than about 10⁻⁸ M and should be preferably greater than about 10⁻⁷ M, about 10⁻⁶ M. Such relatively low catalyst concentrations can be maintained by a variety of methods, such as incorporating in the coating of the implant or the transplant, or in the dressing applied to the would, an osmium containing salt of low solubility, exemplified by the above mentioned Ca, Sr, Ba, Zn or Cu salts. Alternatively, an osmium containing anion or cation can be retained and slowly released from an ion exchange resin or polycationic or polyanionic hydrogel. While the resin in implant and transplant applications would be a hydrated solid matrix, it could be in some applications, such as drops applied to the eardrum or the eye, a liquid. OsO₄, which is soluble both in organic solvents and in water, could be slowly permeating from an organic phase, such as a thermoplastic or elastomeric silicone on the implant, or near the transplant or in the dressing of a wound, or it could be dissolved in a liquid silicone, and applied as an ointment on the skin, at the eye or externally on the eardrum. Alternatively, it could be held by hydrolyzable coordinative or covalent bonds in a hydrogel, and released as the bonds are hydrolyzed, for example by hydration of an osmium cation. Exemplary polymers forming the crosslinked matrix of hydrogels would have osmium weakly complexed, for example to monoamines or to phosphine oxide, to be slowly released. Alternatively, the osmium catalyst could be bound within the hydrogel and decompose the O₂.⁻ diffusing in the hydrogel, protecting, for example, the tissue of the implant coated with the hydrogel. Co-immobilization of the O₂.⁻ catalyst and the OONO⁻ isomerization catalyst in the hydrogel protecting the transplant would be advantageous.

### EXAMPLES

### Example 1

### Os Catalysis, Particularly Os (VIII/VI) Catalysis, of Superoxide Dismutation

Materials and Methods. All chemicals were of analytical grade and were used as received. OsO₄ (4 wt. % solution in water) was purchased from Aldrich (Milwaukee, Wis.), and was freshly diluted before use. K₂OsCl₆.2H₂O Cl₆ and OsCl₃.6H₂O were purchased from Alfa Aesar (Ward Hill, Mass.). Catalase (2 mg/ml, about 130,000 u/ml) was obtained from Boehringer (Mannheim, Germany). Bovine serum albumin (BSA) was purchased from Sigma (St. Louis, Mo.). Peroxynitrite was synthesized, as described elsewhere in detail, by reacting nitrite with acidified H₂O₂ in a quenched-flow system having a computerized syringe pump (WPI Model SP 230IW from World Precision Instruments (Sarasota, Fla.)). 0.63 M nitrite was mixed with 0.60 M H₂O₂ in 0.70 M HClO₄, and the mixture was quenched with 3 M NaOH at room temperature. The stock solution contained 0.11 M peroxynitrite, with about 3% residual H₂O₂ and 12% residual nitrite. The yield of peroxynitrite was determined from its absorption at 302 nm, using ε=1670 M⁻¹cm⁻¹.

Rapid-mixing stopped-flow kinetic measurements were carried out using the Bio SX-17MV Sequential Stopped-Flow from Applied Photophysics (Leatherhead, Surrey, UK) with a 1 cm optical path. The final pH was measured at the outlet of the stopped-flow system in each experiment. All experiments were carried out at 25° C.

Pulse radiolysis experiments were carried out with a Varian (Palo Alto, Calif.) 7715 linear accelerator with 5 MeV electron pulses of 1.5 μs duration. The light source of the analyzing beam was a 200 W xenon lamp. The absorption spectra were measured were at room temperature in a 2 cm Spectrosil® cell, the beam passing the cell three times, for an optical path length of 6.1 cm. The dose was 6-19.4 Gy per pulse, as determined from the absorption of the superoxide ion, using G(O₂.⁻)=6.1 and ε₂₆₀=1940 M⁻¹cm⁻¹, in pH 7.4 O₂ saturated 2.4 mM phosphate buffer, containing 20 mM formate.

OsO₄ does not affect the decay of peroxynitrite. Solutions of 520 μM peroxynitrite in 0.01 M NaOH were mixed with 0.1 M phosphate buffer solutions with and without OsO₄ (0.04 wt. %) at a 1:1 volume ratio to yield a final pH 7.15. The decay of peroxynitrite was followed at 302 nm. It was unaffected by the presence of OsO₄.

OsO₄ (or its product) catalyzes the decay of the superoxide ion radical. The superoxide ion radical (pKₐ=4.8) was formed by pulse-irradiation of oxygenated pH 7.2-7.4 buffer solutions. The solutions contained either 0.02 M formate and 2.4 mM phosphate, or 0.2 M 2-PrOH and 12 mM phosphate. In some experiment 5-10 μM diethylenetriaminepentaacetic acid (DTPA) or catalase (75 u/ml) were added. In the presence of either formate or 2-PrOH. All of the primary radicals formed by the radiation (Equation 3) are converted into O₂.⁻ via Reactions 4-7 when formate is added, and by Reactions 4, 8-10, when 2-PrOH is added. In Equation 3 the values in parentheses are the radiation-chemical yields of the species, defined as the number of species produced per 100 eV of absorbed energy γ.

| | | |
|---|---|---|
| H₂O → e⁻_{aq}(2.6), ^{.}OH (2.7), H^{.} (0.6), | | (3) |
| H₃O⁺ (2.6), H₂O₂ (0.72) | | |
| e⁻_{aq} + O₂ → O₂^{.-} | k₂ = 1.9 × 10¹⁰ M⁻¹s⁻¹ | (4) |
| H^{.} + O₂ → HO₂^{.} | k₃ = 1.2 × 10¹⁰ M⁻¹s⁻¹ | (5) |
| ^{.}OH + HCO₂⁻ → CO₂^{.-} + H₂O | k₄ = 3.2 × 10⁹ M⁻¹s⁻¹ | (6) |
| CO₂^{.-} + O₂ → O₂^{.-} + CO₂ | k₅ = 4.2 × 10⁹ M⁻¹s⁻¹ | (7) |
| ^{.}OH + (CH₃)₂CHOH → (CH₃)₂C^{.}OH + | k₆ = 1.9 × 10⁹ M⁻¹s⁻¹ | (8) |
| H₂O | | |
| (CH₃)₂C^{.}OH + O₂ → (CH₃)₂C(OH)OO^{.} | k₇ = 4.1 × 10⁹ M⁻¹s⁻¹ | (9) |
| (CH₃)₂C(OH)OO^{.} + HPO₄²⁻ → O₂^{.-} + | k₈ = 1.1 × 10⁷ M⁻¹s⁻¹ | (10) |
| (CH₃)₂CO + H₂PO₄⁻ | | |

The decay of O₂.⁻, followed by its absorption at 260 nm, obeyed second-order kinetics in the presence of 10 μM DTPA, with 2k =(5.1±0.1)×10⁵ M⁻¹s⁻¹, in agreement with the earlier reported value of k. DTPA is Usually added in order to chelate metal impurities catalyzing O₂.⁻ dismutation. In the absence of DTPA the decay of O₂.⁻ did indeed deviate from second-order kinetics, and its half-life was about 3-times shorter.

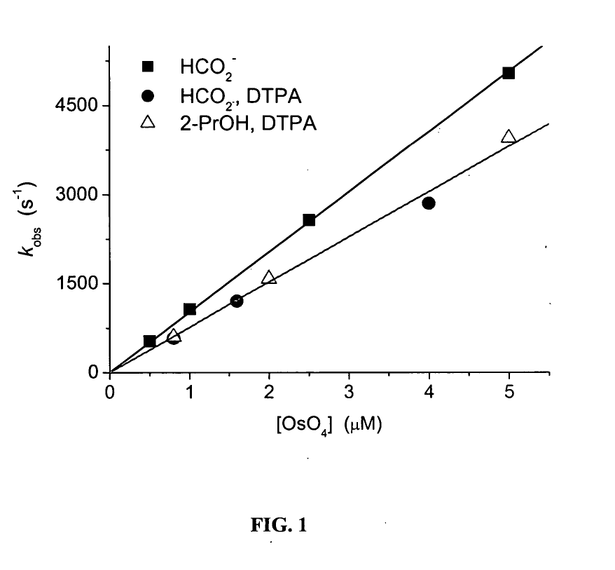

For [O₂.⁻]ₒ>[OsO₄]ₒ, the decay of O₂.⁻ obeyed first-order kinetics and k_{obs} increased linearly with [OsO₄]ₒ (FIG. 1). The rate constant, k_{cat}, for the SOD-mimicking catalytic activity of OsO₄, calculated from the slopes in FIG. 1, was (1.02±0.08)×10⁹ M⁻¹s⁻¹, a value as high as ⅓^{rd} of k_{cat} of copper-zinc superoxide dismutase, CuZn—SOD, the fastest superoxide dismutase. Because the molecular weight of the CuZn—SOD is 32 kDa and that of OsO₄ is only 254 Da, the specific activity of OsO₄ was 42 times higher than that of CuZn—SOD.

Although DTPA usually reduces the activity of dissolved ions catalyzing the dismutation of O₂.⁻, it had only a small effect on the SOD-mimicking activity of OsO₄. When 10 μM DTPA was added to the 20 mM formate-containing solution, k_{cat} dropped only slightly, to (7.6±0.3)×10⁸ M⁻¹s⁻¹. This was also the value of k_{cat} in the presence of 10 μM DTPA when the formate was replaced by 0.2 M 2-PrOH. (FIG. 1).

Low concentrations of OsO₄ were reported to have a catalase-like activity, and the effect of 75 U/mL catalase on the decay of O₂.⁻ has been described. Adding 75 U/mL catalase did not change, however, substantially k_{cat} in our experiments, its value remaining (8.1±0.3)×10⁸ M⁻¹s⁻¹.

Under limiting concentrations of OsO₄, the value of k_{obs} was the same after the 1^{st}, 50^{th} or 100^{th} pulse, showing that OsO₄ (or its product) was not consumed or changed. k_{obs} was unaffected by the number of pulses delivered to any of the above-described solutions.

To test the stability of the catalyst solution upon storage, OsO₄ (5 μM) was stored in the pH 7.25 20 mM formate solution and in the 0.2 M 2-PrOH-solution. At neutral pH and at 25° C. formate and 2-PrOH were only slowly oxidized by the catalyst. After 4 days, k_{cat} was (7.3±0.3)×10⁸ for the formate and (4.4±0.2)×10⁸ M⁻¹s⁻¹ for the 2-PrOH solution. In the 1-2 hour long experiments, the concentration of OsO₄ or its catalytic product was practically unchanged in either solution. OsO₄ or its catalytic product was fairly stable in 20 mM formate or in 0.2 M 2-PrOH and maintained its catalytic activity when 10 μM DTPA was added.

It has been suggested that catalysis of O₂.⁻ dismutation by SOD, as well as that by other organic and metallo-organic compounds proceeds via a “ping-pong” mechanism, the catalyst oscillating between two oxidation states. (Equations 11 and 12)
Osⁿ⁺+O₂.⁻→Os⁽ⁿ⁻¹⁾⁺+O₂ ( 1)
Os⁽ⁿ⁻¹⁾⁺+O₂.⁻+2H⁺→Osⁿ⁺+H₂O₂ (12)

The dismutation rate is given by Equation 13 assuming the steady-state approximation for Osⁿ⁺ and Os⁽ⁿ⁻¹⁾⁺.
−d[O₂.⁻]/dt=2k₉k₁₀/(k₉+k₁₀)[Osⁿ⁺]₀[O₂.⁻]=k_{cat}[Osⁿ⁺]₀[O₂.⁻] (13)

When the rate limiting constituent was not OsO₄ or its derivatives but O₂.⁻, its decay obeyed first order kinetics and k_{obs} increased linearly with [OsO₄]ₒ, with k₉=(1.3±0.1)×10⁹ M⁻¹s⁻¹. Because k_{cat}=(1.02±0.08)×10⁹ M⁻¹s⁻¹, k₁₀=(8.7±0.3)×10⁸ M⁻¹s⁻¹.

When the concentration of OsO₄ exceeded that of O₂.⁻, a transient species having an absorption maximum at 310 nm was observed (ε₃₁₀=2050±150 M⁻¹cm⁻¹). The species decayed via a second-order reaction, with k=(2.0±0.3)×10⁵ M⁻¹s⁻¹, which did not depend on the intensity of the pulse or on [OsO₄]ₒ. We propose that the transient species is Os⁽ⁿ⁻¹⁾⁺ (Reactions 14, 15, 15a) or the ion pair Osⁿ⁺O₂.⁻ (Reaction 16). Under catalytic conditions, i.e., [Osⁿ⁺]ₒ<[O₂.⁻]ₒ, Os⁽ⁿ⁻¹⁾⁺ or Os⁽ⁿ⁻¹⁾⁺O₂.⁻ react with O₂. to form H₂O₂ through Reaction 15 or 15a, respectively, whereas under non-catalytic conditions it decomposes in a bi-molecular reaction (Reaction 16).
Osⁿ⁺+O₂.⁻→Os—O₂⁽ⁿ⁻¹⁾⁺ (14)
Os⁽ⁿ⁻¹⁾⁺(or Os—O₂⁽ⁿ⁻¹⁾⁺)+O₂.⁻+2H⁺→Osⁿ⁺+H₂O₂ (or +O₂) (15)
2 Os⁽ⁿ⁻¹⁾⁺→Osⁿ⁺+Os⁽ⁿ⁻²⁾⁺(or +2O₂) (15a)
or
2Os—O₂⁽ⁿ⁻¹⁾⁺→Osⁿ⁺+Os⁽ⁿ⁻²⁾⁺(or +2O₂) (16)

In order to identify the redox species participating in the “ping-pong” sequence of Reactions 11 and 12, we studied the effect of Os^{III}, Os^{IV}, and Os^{VI} on the decay of O₂.⁻.

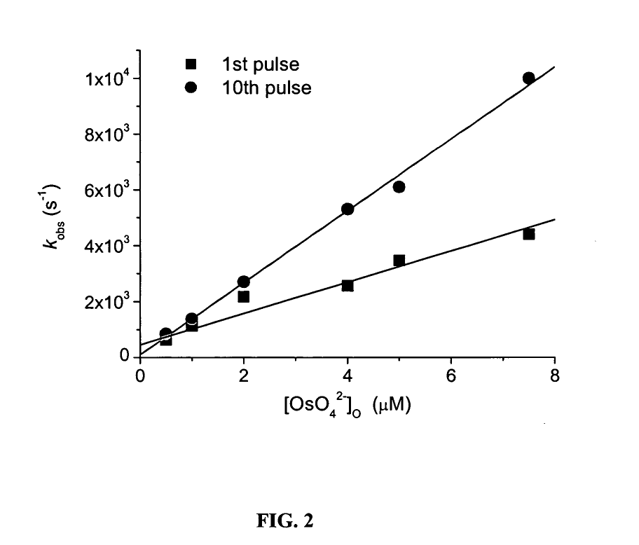

The decay of 10 μM O₂.⁻ was followed upon pulse-irradiation of oxygenated solutions containing 20 mM formate, 2.4 mM phosphate buffer (pH 7.25) and 1.65 or 3.3 μM OsCl₃. OsCl₃ itself was a poor catalyst, but upon repeated pulsing it was converted into a good one. In the first pulse, the adding of OsCl₃ caused only a minor increase in the rate decay of O₂.⁻, but k_{obs} increased enormously upon repetitive pulsing, reaching a plateau after 40 pulses. At the plateau k_{obs}=1.7×10³ and 3.4×10³ s⁻¹ in the presence of 1.65 and 3.3 OsCl₃, respectively, resulting in k_{cat} =1.1×10⁹ M⁻¹s⁻¹, a value within experimental error of that obtained when OsO₄was added, k_{cat}=(1.02±0.08)×10⁹ M⁻¹s⁻¹. When the same experiment was carried out in the presence of 5 μM OsCl₆²⁻, the system behaved similarly, the decay of O₂.⁻ being enhanced upon repetitive pulsing, but reaching a plateau of only k_{cat}=4×10⁸ M⁻¹s⁻¹. In the case of OsO₄²⁻, k_{cat} obtained by the 1^{st} pulse was about 60% lower than that obtained after the 10^{th}, i.e., k_{cat}(1)=(5.6±0.6)×10⁸ M⁻s⁻¹ and k_{cat}(10)=(1.3±0.1)×10⁹ M⁻¹s⁻¹ (FIG. 2).

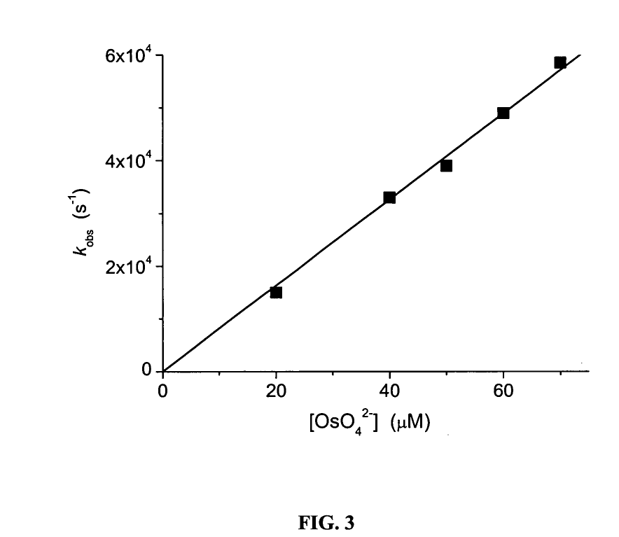

In the presence of excess of Os(VI) over O₂.⁻, i.e., non-catalytic conditions, the formation of the same transient species formed under non-catalytic conditions using OsO₄ was observed. The decay of ca. 4 μM O₂.⁻ was linearly dependent on [OsO₄²⁻]ₒ resulting in k₉=(8.2×0.1)×10⁸ M⁻¹s⁻¹ (FIG. 3).

These results suggest that the radiolytically generated O₂.⁻ and H₂O₂ (formed through the dismutation of O₂.⁻ and by the pulse (Equation 3)) oxidize Os^{III}, Os^{IV} and Os^{VI} till the most efficient redox couple is achieved, i.e., Os^{VII}/Os^{VIII}.

An important transient species is Os^{VII} (Reaction 17). Under catalytic conditions, i.e., [Os^{VIII}]ₒ<[O₂.⁻]ₒ, Os^{VII} reacts with O₂.⁻ to form H₂O₂ through Reaction 15, whereas under non-catalytic conditions it decays in a bi-molecular reaction (Reaction 15a or 16, particularly Reaction 17).
2 Os^{VII}→Os^{VIII}+Os^{VI} (17)

### Example 2

### Catalysis of the Decomposition of Carbonate Radical Anion

Materials. Poly-4-vinylpyridine N-oxide (4-PVPNO), ˜200 kD solid and Poly-2-vinylpyridine N-oxide (2-PVPNO), ˜300 kD solid were purchased from Polysciences, Warrington, Pa. The lower molecular weight, 4-PVPNO, Reilline™ 4140 (40% aqueous solution), was purchased from Reilly Industries, Indianapolis, Ind. 4-picoline N-oxide (98%) was purchased from Sigma-Aldrich, St. Louis, Mo.

Methods. Pulse radiolysis experiments were carried out using a 5-MeV Varian 7715 linear accelerator (0.05-1.5 μs electron pulses, 200 mA current). All measurements were performed at room temperature in a 2-cm spectrosil cell, with three light passes (optical path length 6.1 cm). The formation and decay kinetics of the CO₃₋ radical were tracked by measuring its absorption at 600 nm using ε₆₀₀=1860 M⁻¹cm⁻¹.

Carbonate radical was generated by irradiating N₂O-saturated (˜25 mM) aqueous solutions containing 0.1-0.6 M sodium carbonate (pKₐ(HCO₃⁻)=10, I=0.5 M) at pH 10.0 by reaction sequence 18-20 (the species radiation yields are in parentheses):

| | | |
|---|---|---|
| H₂O → e⁻_{aq}(2.6), ^{.}OH (2.7), H^{.} (0.6), | | (18) |
| H₃O⁺ (2.6) | | |
| e⁻_{aq} + N₂O → N₂ + OH⁻ + ^{.}OH | k₁₉ = 9.1 × 10⁹ M⁻¹s⁻¹ | (19) |
| ^{.}OH + CO₃²⁻ → CO₃^{.}— + OH⁻ | k₂₀ = 3.9 × 10⁸ M⁻¹s⁻¹ | (20) |
| ^{.}OH + HCO₃⁻ → CO₃^{.}— + H₂O | k₂₀ₐ = 8.5 × 10⁶ M⁻¹s⁻¹ | (20a) |

In the absence of any added substrate, the decay of CO₃.— was second-order with 2k₂₁=(2.3±0.3)×10⁷, (2.9±0.3)×10⁷ and (3.8±0.4)×10⁷ M⁻¹s⁻¹ in the presence of 0.1, 0.2 and 0.6 M carbonate, respectively.
CO₃.—+CO₃.—→products (21)

The addition of 2 mM 4-picoline N-oxide shortened the half-life of 3 μM CO₃.— by about 50% in the presence of 0.6 M carbonate. A high concentration of carbonate was used to avoid the reaction of 4-picoline N-oxide with .OH radicals (k=3×10⁹ M⁻¹s⁻¹ Neta et al. J. Phys. Chem. 1980, 84, 532-4). The rate constant of the reaction of CO₃.— with 4-picoline N-oxide was about 3×10⁴ M⁻¹s⁻¹.

The yield of CO₃.— remained unchanged when any of the three PVPNOs was added, showing that the carbonate ions scavenged all of the .OH produced. This is quite surprising because .OH adds rapidly to pyridine N-oxide or to its methylated derivatives, 2, 3 or 4-picoline N-oxide (k=3×10⁹ M⁻¹s⁻¹). The highest PVPNO concentration in the experiments was 0.4%, corresponding to a mer concentration of about 32 mM versus about 100 mM CO₃²; yet the .OH radicals were scavenged by the carbonate ions, not by PVPNO, proving that the rate constant for the reaction of .OH with an average mer of PVPNO was less than about 1×10⁸ M⁻¹s⁻¹.

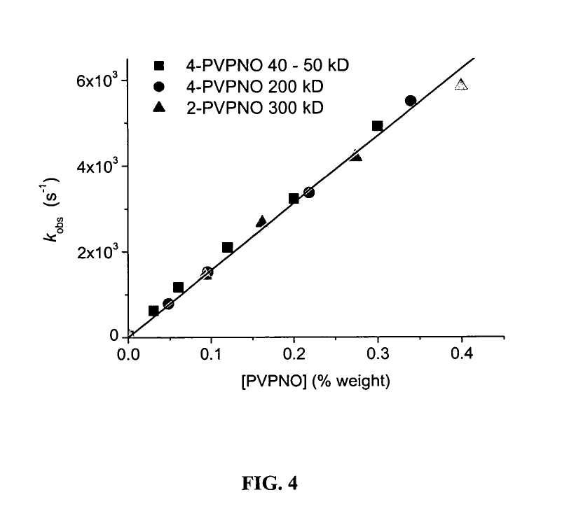

The rate of decay of CO₃.— was, however, most drastically enhanced when any of the three PVPNOs were added. The decay kinetics changed from second-order to first-order and k_{obs} increased upon increasing the PVPNO concentration. (FIG. 4)

Evidently, the rapid decay was caused by the reaction of PVPNO with CO₃.— (Reaction 22).
CO₃.—+PVPNO products (22)

The bimolecular rate constant for the PVPNO, when its concentration was expressed in weight %, was very high and it was independent of the type and molecular weight of the PVPNO. Thus, k₂₂=(7.0±0.8)×10⁷, (3.1±0.2)×10⁸ and (4.7±0.2)×10⁸ M⁻¹s⁻¹ for the Reilline™ and 200 kD 4-PVPNOs and 2-PVPNO, respectively, assuming a MW of 45±5 kD for the Reilline™ PVPNO.

The decay rate of CO₃.— was barely affected by repetitively applying as many as 300 pulses, generating 1.5 mM CO₃.—, when the PVPNO concentrations were >0.1 weight %, proving that the polymer was a superior scavenger of CO₃.—.

The radiolytically produced H₂O₂ had no effect on the results. The results were also unchanged when the solution used contained an initial 0.12 mM concentration of H₂O₂.

The absence of rapid consumption of PVPNO in the series of experiments performed suggests that at least some, probably most, and possibly all of the oxidized radical lesions, created when CO₃.— radicals captured electrons from, or injected holes into, PVPNO, were repaired. Repair of the lesions makes PVPNO a catalyst for the decomposition of CO₃.⁻.

We note that in protonated PVPNO, the nitrogen atoms of the pyridinium rings have OH-functions. They resemble in this respect cyclic hydroxylamines (RNO—H), known to react rapidly with CO₃.— to form nitroxides, RNO. The nitroxides react further, even faster, with CO₃.—, to form oxoammonium cations, RN⁺═O, the decomposition of which is base-catalyzed.

While the above is a complete description of the preferred embodiments of the invention, various alternatives, modifications, and equivalents may be used. Therefore, the above description should not be taken as limiting the scope of the invention which is defined by the appended claims.

1. An implant, transplant, or dressing containing a pharmaceutically acceptable osmium compound.

2. An implant, transplant, or dressing as in claim 1 which releases the osmium compound in a controlled manner.

3. An implant, transplant, or dressing according to claim 1, where the nominal valence of the osmium compound is at least four.

4. An implant, transplant, or dressing according to claim 3, where the nominal valence of the osmium compound is at least six.

5. An implant, transplant, or dressing according to claim 4, where the nominal valence of the osmium compound is eight.

6. An implant, transplant, or dressing according to claim 1, where at least two of the atoms neighboring the osmium atom of the compound are oxygen atoms.

7. An implant, transplant, or dressing according to claim 6, where at least three of the atoms neighboring the osmium atom of the compound are oxygen atoms.

8. An implant, transplant, or dressing according to claim 7, where at least four of the atoms neighboring the osmium atom of the compound are oxygen atoms.

9. An implant, transplant, or dressing according to claim 1, where the implant comprises a stent.

10. A stent, according to claim 9, where the stent comprises a vascular stent.

11. A stent, according to claim 10, where the stent comprises a coronary stent.

12. An implant, transplant, or dressing according to claim 1, where the implant comprises a vascular implant.

13. An implant, transplant, or dressing according to claim 1, where the implant comprises an orthopedic implant.

14. An implant, transplant, or dressing according to claim 1, where the implant comprises a cosmetic implant.

15. An implant, transplant, or dressing according to claim 1, where the implant comprises a sack containing living cells.

16. An implant, transplant, or dressing according to claim 1, where the implant comprises a cochlear implant.

17. An implant, transplant, or dressing according to claim 1, where the implant comprises a device which monitors temperature, or flow, or pressure, or the concentration of a chemical, or a biochemical, or any combination of these.

18. An implant, transplant, or dressing according to any of claims 1-8, where the osmium compound is bound within a hydrogel.

19. An implant, transplant, or dressing according to any of claims 1-8, where the osmium compound is bound within polycation.

20. An implant, transplant, or dressing according to any of claims 1-8, where the osmium compound is bound within polyanion.

21. An anti-inflammatory topical composition containing a pharmaceutically acceptable osmium compound.

22. The anti-inflammatory topical composition of claim 21, releasing in a controlled manner an osmium compound.

23. The anti-inflammatory topical composition of claim 21, formulated for use on the skin or in the ear.

24. The anti-inflammatory topical composition of claim 21, where the nominal valence of the osmium compound equals, or is greater than, four.

25. The anti-inflammatory topical composition according to claim 24, where the nominal valence of the osmium compound equals, or is greater than, six.

26. The anti-inflammatory topical composition according to claim 25, where the nominal valence of the osmium compound is eight.

27. The anti-inflammatory topical composition according to any of claims 21 to 26, where at least two of the atoms neighboring the osmium atom of the compound are oxygen atoms.

28. The anti-inflammatory topical composition according to claim 27, where at least three of the atoms neighboring the osmium atom of the compound are oxygen atoms.

29. The anti-inflammatory topical composition according to claim 28, where at least four of the atoms neighboring the osmium atom of the compound are oxygen atoms.

30. An osmium compound containing pharmaceutically acceptable composition containing osmium for treatment of a disease associated with, or resulting of, superoxide dismutase deficiency.

31. A pharmaceutically acceptable composition containing an osmium compound for treatment of a disease associated with, or resulting from, one or more mutations or defects in a superoxide dismutase.

32. A pharmaceutically acceptable composition according to claim 31 for the treatment of a condition selected for the group consisting of neurodegenerative disorder, an autoimmune disease, an alcoholic liver disorder, an arthritic disease, Peyronie's disease, cardiovascular disease, an inflammatory bowel disease, Crohn's disease, scleroderma, dermatitis, and Lou Gehrig's disease.

33. A pharmaceutically acceptable compound according to claim 31, where the nominal valence of the osmium compound equals, or is greater than, four.

34. A pharmaceutically acceptable compound according to claim 33, where the nominal valence of the osmium compound equals, or is greater than, six.

35. A pharmaceutically acceptable compound according to claim 34, where the nominal valence of the osmium compound is eight.

36. A pharmaceutically acceptable compound according to claims 35, where at least two of the atoms neighboring the osmium atom of the compound are oxygen atoms.

37. A pharmaceutically acceptable compound according to claim 36, where at least three of the atoms neighboring the osmium atom of the compound are oxygen atoms.

38. A pharmaceutically acceptable compound according to claim 37, where at least four of the atoms neighboring the osmium atom of the compound are oxygen atoms.

39. A method for prevention or treatment of adverse inflammation comprising administering to a patient by an osmium containing superoxide decay accelerating compound wherein the concentration of the osmium compound delivered to or near a treated tissue or organ is in the range from 10⁻⁶M to 10⁻¹⁰M.

40. A method according to claim 34, wherein the concentration of the osmium compound is in the range from 10⁻⁷M to 10⁻⁹M.

41. A method as in any of claims 39 and 40, wherein the patient suffers from a condition selected from the group consisting of neurodegenerative disorder, an autoimmune disease, an alcoholic liver disorder, an arthritic disease, Peyronie's disease, cardiovascular disease, an inflammatory bowel disease, Crohn's disease, scleroderma, dermatitis, and Lou Gehrig's disease.

42. An implant or a transplant comprising a polymer having N-oxide functions.

43. An implant or transplant according to claim 42, where the N-oxide pyridine-N-oxide or a derivative of pyridine N-oxide.

44. An implant or transplant according to claim 42, where the polymer comprises a poly(vinylpyridine-N-oxide).

45. An implant or transplant according to claim 43 wherein the polymer comprises poly(2-vinylpyridine-N-oxide).

46. An implant or according to any of claims 42-44, wherein the implant is a stent.

47. A stent according to claim 46, wherein the stent is a vascular stent.

48. A vascular stent according to claim 46, wherein the vascular stent is a coronary stent, a kidney, pancreatic islets or Langerhans cells, a heart, a bone, skin, blood vessel, liver, or lung.

49. An implant, transplant, or dressing containing a pharmaceutically acceptable osmium compound and a polymer having N-oxide functions.
